(19) United States
(12) Patent Application Publication
(10) Pub. No.: US 2014/0005280 A1
(43) Pub. Date: Jan. 2, 2014

(54) CARBOXYMETHYL STARCH AND CHITOSAN POLYELECTROLYTE COMPLEXES

(71) Applicants: Elias Assaad, Montreal (CA); Mircea-Alexandru Mateescu, Montreal (CA)

(72) Inventors: Elias Assaad, Montreal (CA); Mircea-Alexandru Mateescu, Montreal (CA)

(73) Assignee: 4413261 Canada Inc. (Spencer Canada), Alcove (La Peche ) (CA)

(21) Appl. No.: 13/980,518

(22) PCT Filed: Jan. 19, 2012
(86) PCT No.: PCT/CA2012/000061

§ 371 Date: Sep. 18, 2013

Related U.S. Application Data
(60) Provisional application No. 61/434,142, filed on Jan. 19, 2011.

(51) Int. Cl. A61K 47/36; C08L 3/08

(52) U.S. Cl. CPC A61K 47/36; C08L 3/08
USPC 514/778; 536/50

(57) ABSTRACT

A carboxymethyl starch and chitosan polyelectrolyte complex is provided. A carrier comprising this polyelectrolyte complex along with a solid oral dosage form comprising this polyelectrolyte complex are also provided. A method of manufacturing the polyelectrolyte complex is also provided.

### CROSS REFERENCE TO RELATED APPLICATIONS

This application claims priority of U.S. provisional patent application 61/434,142, filed on Jan. 19, 2011 the specifications of which is hereby incorporated by reference.

### FIELD OF THE INVENTION

The present invention relates to carboxymethyl starch and chitosan polyelectrolyte complexes, as well as uses thereof for example as active ingredient carriers.

### BACKGROUND OF THE INVENTION

Since carboxymethyl starch (CMS) was proposed as an excipient for controlled drug release from oral solid dosage forms (tablet), several studies have been undertaken in order to investigate the properties and the efficiency of this excipient. The influence of the degree of substitution (DS), of the degree of protonation, and of the formulated drug type and loading on release kinetics of small molecules from CMS matrices has been recently studied. Moreover, the effects of certain formulation parameters, such as compression force and NaCl electrolyte particle size, on the rate of drug release have been investigated. CMS has also been suggested for the formulation of large size bioactive agents, such as pancreatic enzymes (α-amylase, lipase and trypsin), Escherichia coli, filamentous surface proteins of Escherichia coli (F4 fimbriae) and Lactobacillus rhamnosus probiotic (Calinescu & Mateescu, 2008). These studies have shown that CMS can reduce the damaging effect of the acidity of the gastric medium on bioactive agents and affords a controlled drug release in the intestinal medium. In simulated gastric fluid (SGF, pH 1.2), the CMS in the outer layer of tablet is protonated, making the matrix compact. At higher pH (simulated intestinal fluid, SIF, pH 6.8), the carboxyl groups are deprotonated and ionized, thus favoring hydration, swelling and finally solubilisation of tablet. The solubility of CMS in neutral medium (SIF) and its digestion by pancreatic α-amylase can be limiting factors to effect a sustained drug release (Calinescu & Mateescu, 2008).

With the aim to ensure a longer time of drug release and targeting to the colon, chitosan dry powder has been used as a coexcipient in such formulations (Calinescu & Mateescu, 2008). Chitosan has been shown to interact with unmodified starch via intermolecular hydrogen bonds, leading to the formation of chitosan-starch complex (Xu, Kim, Hanna & Nag, 2005).

Chitosan has been found to interact with carboxymethyl starch (CMS) to form nano-sized particles (Saboktakin, Tabatabaei, Maharramov & Ramazanov, 2010).

Therefore, there remains a need for alternative formulation of carboxymethyl starch and chitosan, such as monolithic devices (tablets, implants, prills, and pellets).

Furthermore, there remains a need for alternative formulations of carboxymethyl starch and chitosan for the preparation of drug delivery vehicles.

### SUMMARY OF THE INVENTION

According to an embodiment, there is provided a carboxymethyl starch and chitosan polyelectrolyte complex, wherein the carboxymethyl starch is a high amylose carboxymethyl starch.

The carboxymethyl starch may be a carboxymethyl starch salt.

The carboxymethyl starch salt may be sodium carboxymethyl starch or potassium carboxymethyl starch.

The carboxymethyl starch salt may be sodium carboxymethyl starch.

The carboxymethyl starch salt may be partially protonated.

The degree of substitution of the carboxymethyl starch may be between about 0.03 and about 2.

The degree of substitution of the carboxymethyl starch may be about 0.14.

The degree of deacetylation of the chitosan may be about 65% or more.

The degree of deacetylation of the chitosan may be about 80%.

The molecular weight of the chitosan may be about 100 kDa or more.

The molecular weight of the chitosan may be between about 400 and about 700 kDa.

The molecular weight of the chitosan may be about 700 kDa.

The —NH₃⁺ groups of the chitosan and —COO⁻ groups of the carboxymethyl starch may be present in a (—NH₃⁺:—COO⁻) ratio ranging from about to about 1:0.5 to about 0.5:1.

The —NH₃⁺ groups of the chitosan and the —COO⁻ groups of the carboxymethyl starch may be present in a 1:1 (—NH₃+:—COO⁻) ratio.

The polyelectrolyte complex may be an active ingredient carrier.

The active ingredient carrier may be comprised within a solid oral dosage form.

The polyelectrolyte complex may be for use as an active ingredient carrier.

The active ingredient carrier may be for use in a solid oral dosage form.

The polyelectrolyte complex of any one of claims 1 to 18, wherein the polyelectrolyte complex is solid.

According to another embodiment, there is provided an active ingredient carrier comprising the polyelectrolyte complex of the present invention.

According to another embodiment, there is provided a solid oral dosage form comprising the polyelectrolyte complex of the present invention and an active ingredient.

The dosage form may be further comprising an additional pharmaceutically acceptable excipient.

According to another embodiment, there is provided a method of manufacturing a carboxymethyl starch and chitosan polyelectrolyte complex, the method comprising coagulating together carboxymethyl starch and chitosan in a solvent.

The coagulating may be carried out in an aqueous medium.

The coagulating may be carried out by mixing an aqueous solution of carboxymethyl starch and an aqueous solution of chitosan.

According to another embodiment, there is provided a method of manufacturing a carboxymethyl starch and chitosan polyelectrolyte complex, comprising coagulating together carboxymethyl starch and chitosan in a solvent, wherein the carboxymethyl starch and the chitosan are as defined in the present invention.

The coagulation may be carried out at a (—NH₃⁺:—COO⁻) ratio ranging from about 1:0.5 to about 0.5:1.

The coagulation may be carried out at a 1:1 (—NH₃⁺:—COO⁻) ratio.

The method may be further comprising isolating the polyelectrolyte complex.

The method may be further comprising washing and drying the polyelectrolyte complex.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates scanning electron microscopy micrographs of (a) CMS, (b) chitosan-400, (c) chitosan-700, and (d) polyelectrolyte complex (PEC) at magnification of 100× (labelled as a1, b1, c1, d1) and 500× (labelled as a2, b2, c2, d2) and voltage of 15 kV.

FIG. 2 illustrates FTIR spectra of CMS, chitosan-700, 50% CMS:50% chitosan-700, and PEC. Pellets (12 mm diameter) are prepared by compression at 3 tonnes of KBr (67 mg) and sample (3 mg) mixtures.

FIG. 3 illustrates X-ray diffraction patterns of CMS, chitosan-700, and PEC.

FIG. 4 illustrates thermogravimetric patterns of CMS, chitosan-700, 50% CMS:50% chitosan-700, and PEC at a heating rate of 10° C./min between 25 and 600° C.

FIG. 5 illustrates NMR images at various times of unloaded tablets of (CMS, chitosan-400, chitosan-700, 50% CMS:50% chitosan-700 and PEC) incubated for 2 h in SGF and then transferred to SIF: (A) axial side images, (B) axial and radial swelling. (x) indicates the radial direction and (y) the axial direction.

FIG. 6 illustrates photographs of CMS, chitosan-700, 50% CMS 50% chitosan-700 and PEC tablets (200 mg, 20% loading) during dissolution tests (1 L, 37° C., 100 rpm). Photographs are taken for the tablets, first after 2 h of incubation in SGF and then after the complete drug (acetaminophen or aspirin) release in SIF. The sizes of tablets are not normalized.

FIG. 7 illustrates the kinetics of drug dissolution from tablets (200 mg, 20% loading) of CMS, chitosan-400, chitosan-700, 50% CMS:50% chitosan-400, 50% CMS:50% chitosan-700 and PEC. The tablets are incubated (1 L, 37° C., 100 rpm) for 2 h in SGF and then transferred to SIF. A) acetaminophen; B) metformin, monolithic tablets are incubated only in SGF; C) aspirin.

### DETAILED DESCRIPTION OF THE PREFERRED EMBODIMENTS

Turning to the present invention in more detail, there is provided a carboxymethyl starch and chitosan polyelectrolyte complex and uses thereof.

As used herein, “polyelectrolyte complex” refers a chemical complex of polyelectrolytes.

A polyelectrolyte is a polymer whose repeating units, or some of them bear an electrolyte group. Such groups will dissociate in aqueous solution (such as water), making the polymer charged [charged polymers are also called polyions, polycations (positively charged polymers), and polyanions (negatively charged polymers)].

A polyelectrolyte complex (PEC) is formed by oppositely charged polyelectrolytes. More specifically, a polyelectrolyte complex is formed through electrostatic interactions between the positive charges of a polycation and the negative charges of a polyanion. Hydrogen bonding may also play a more or less important role in the formation of the complex. Typically, when a polycation and a polyanion are mixed together in an aqueous solution, a polyelectrolyte complex forms due to the strong interactions between them. These interactions lead to the formation of the complex (in essence a new molecule) where the polyanion and the polycation are bonded together through electrostatic interactions and also possibly hydrogen bonds.

The formation of a polyelectrolyte complex from a polyanion and a polycation is often easy to assess visually. Indeed, in the case where the polyanion is in solution in a solvent (often an aqueous solvent), the polycation is also in solution in a solvent (again often an aqueous solvent) and both solutions are mixed together, complex formation is often evidenced by a thickening effect, coagulation, jellification and/or PEC precipitation. Thus, starting from two solutions of polyelectrolytes, mixing results in thickening, coagulation, jellification and/or precipitation due to the fact that the formed polyelectrolyte complex is less soluble than the separate polyanion and polycation. Polyelectrolyte complex formation can be seen as a self-assembly process by which a polysalt is produced. As such, a polyelectrolyte complex is different from a simple mixture of its constituent polyelectrolytes. It is a different chemical entity with different characteristics, such as morphology, density, solubility, and XRD pattern (order degree), as elaborated in Example 1 below.

Chitosan is a linear polysaccharide composed of randomly distributed β-(1-4)-linked D-glucosamine (deacetylated unit,

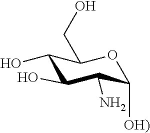

and N-acetyl-D-glucosamine (acetylated unit,

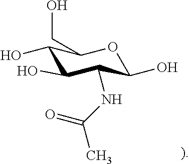

It has a number of commercial and possible biomedical uses. Chitosan is produced commercially by deacetylation of chitin, which is the structural element in the exoskeleton of crustaceans and cell walls of fungi. The degree of deacetylation of chitosan can be determined by various techniques, including acid-base titration and NMR spectroscopy.

Carboxymethyl starch (CMS) is a modified starch. Starch is a polysaccharide produced by all green plants as a seed energy store. More specifically, starch is a carbohydrate consisting of a large number of glucose units joined together by glycosidic bonds. Starch consists of two types of molecules: the nonbranched helical amylose and the branched amylopectin. The proportion of these two molecules in any given starch depends on the plant from which the starch originates. Typically, a starch comprises between about 20 and about 25% amylose.

Carboxymethyl starch is a starch in which the hydroxy groups on some of the glucose units are replaced by carboxymethyl groups:

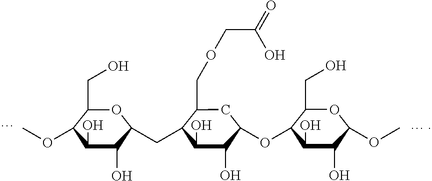

The degree of substitution by these carboxymethyl groups can be measured by various techniques including back-titration.

In the present invention, the starch can be from any origin, non-limiting examples of which include corn, potato, wheat, rice, etc.

In embodiments of the present invention, the polyelectrolyte complex of the invention is in solid form (i.e., not in the form of a solution or a gel; it rather is a solid such as a powder).

In embodiments, the carboxymethyl starch in the polyelectrolyte complex is a high amylose carboxymethyl starch. As stated above, plants generally produce starch comprising between about 20 and about 25% amylose. However, some plants, like certain species of maize (corn), produce starches having more than 50% amylose. Therefore, herein, a “high amylose starch” is a starch comprising more than 50% amylose. For example, a high amylose starch can comprise between about 50% and about 90% amylose. In embodiments of the present invention, the carboxymethyl starch comprises about 50%, 60%, 70%, 80% or 90% amylose or more and/or less than about 90%, 80%, 70% or 60% amylose (while at least comprising at least 50% amylose). In embodiments, the carboxymethyl starch comprises between about 50% and about 60%, or between about 50% and about 70%, or between about 50% and about 80%, or between about 50% and about 90%, or between about 60% and about 70%, or between about 60% and about 80% amylose, or between about 60% and about 90%, or between about 70% and about 80%, or between about 70% and about 90%, or between about 80% and about 90%, or about 70% amylose.

In embodiments, the carboxymethyl starch is in the form of sodium carboxymethyl starch. This means that sodium carboxymethyl starch, carboxymethyl starch as a polysalt with sodium counterions, is used in forming the polyelectrolyte complex. It is to be understood that many, if not all, of the sodium counterions may be replaced by counterions from the chitosan upon formation of the polyelectrolyte complex. It is expected however that some sodium counterions may remain in the polyelectrolyte complex. Other carboxymethyl starch salts can be used in the present invention. Non-limiting examples of such salt includes potassium salt. Also, in embodiments, partially protonated carboxymethyl starch, with —COOH groups instead —COONa (or other such as —COOK), is used to prepare the polyelectrolyte complex.

In embodiments, the degree of substitution of the carboxymethyl starch is between about 0.03 and about 2. In more specific embodiments, the degree of substitution is between about 0.05 and about 1, between about 0.05 and about 0.5, between about 0.05 and about 0.2, between about 0.1 and about 0.2, or about 0.14. In embodiments, the degree of substitution is about 0.03, 0.05, 0.1, 0.5, 0.8, 1, 1.2, 1.5, 1.8 or more and/or is about 2, 1.8, 1.5, 1.2, 1, 0.8, 0.5, or 0.2, or less. For certainty, a degree of substitution of, for example, 0.14 means that, on average, 0.14 hydroxy group per glucose unit has been replaced by a carboxymethyl group.

The molecular weight of the carboxymethyl starch is particularly not limited. In embodiments, the molecular weight is about 50, 100, or 125 kDa or more and/or about 200, 150, or 125 kDa or less. In embodiments, the molecular weight ranges from about 100 kDa to about 150 kDa.

In embodiments, the degree of deacetylation of chitosan is about 65%, 75% or 80% or more and/or about 90%, 80%, 70% or less. In yet another embodiment, the degree of deacetylation is about 80% or more. In more specific embodiments, the degree of deacetylation is about 80%. For certainty, a degree of substitution of 80% means that 80% of repeating units of the chitosan are D-glucosamine (rather than N-acetyl-D-glucosamine).

In embodiments, the molecular weight of the chitosan is about 100, 200, 300, 400, 500, 500, 600, or 700 kDa or more and/or 700, 600, 500, 400, 300, 200, 100 kDa or less. In further embodiments, the molecular weight of the chitosan is between about 400 kDa and about 500 kDa, or between about 400 kDa and about 600 kDa, or between about 400 kDa and about 700 kDa, and in a further embodiment, it is about 700 kDa.

In embodiments, the —NH₃⁺ groups of the chitosan and the —COO⁻ groups of the carboxymethyl starch are present in a (—NH₃⁺:—COO⁻) ratio ranging from about 1:0.5 to about 0.5:1, i.e. in embodiments there may be some —NH₃⁺ groups or some —COO⁻ groups in excess. In further embodiments, the —NH₃⁺ groups of the chitosan and —COO⁻ groups of the carboxymethyl starch are present in a (—NH₃⁺:—COO⁻) ratio ranging from about 1:0.8 to about 0.8:1, or in about a 1:1 (—NH₃⁺:—COO⁻) ratio. In embodiments, the —NH₃⁺ groups of the chitosan and the —COO⁻ groups of the carboxymethyl starch are present in the following (—NH₃⁺:—COO⁻) ratio: 1:0.5, 1:0.6, 1:0.7, 1:0.8, 1:0.9, 1:1, 0.9:1, 0.8:1, 0.7:1, 0.6:1 or less and/or 0.5:1, 0.6:1, 0.7:1, 0.8:1, 0.9:1, 1:1, 1:0.9, 1:0.8, 1:0.7, 1:0.6, or more.

It is to be noted that a 1:1 (—NH₃⁺:—COO⁻) ratio corresponds to about 14% (w/w) of chitosan in the polyelectrolyte complex (based on the total weight of the polyelectrolyte complex). If a lower (—NH₃⁺:—COO⁻) ratio is used, the polyelectrolyte complex will comprise less chitosan (for example as little as about 7.5% for a 0.5:1 ratio). If a higher (—NH₃⁺:—COO⁻) ratio is used, the polyelectrolyte complex will comprise more chitosan (for example as much as 24.6% for a 1:0.5 ratio).

The above-described complex is useful for example as a carrier for active ingredients, for example in solid oral dosage forms. Therefore, the present invention is also concerned with carriers for active ingredients comprising the above polyelectrolyte complex as well solid oral dosage forms comprising this polyelectrolyte complex.

As used herein, carriers for active ingredients is an excipient useful for formulating an active ingredient in a dosage form. The polyelectrolyte complex of the invention is particularly useful as an active ingredient carrier in applications where release of the active ingredient in the colon is desired (see Example 1).

A solid oral dosage form is a dosage form comprising an active ingredient administrable orally and in solid form. Examples of solid oral dosage form (e.g., monolithic formulations) include tablets (coated or not), capsules (containing particles), dragées, etc.

In embodiments, the solid oral dosage form is a coated or uncoated compressed tablet. In embodiments, these tablets can consist of the active ingredient(s) and the polyelectrolyte complex only. In other embodiments, the tablet can also comprise other pharmaceutically acceptable excipients (non-active ingredients). Such excipients include diluents, binders, lubricants, glidants, disintegrants, coloring agents, flavoring agents and the like, as described below. Such excipients should be non-toxic. Non-toxic excipients are well known to the skilled person and have been described in numerous publications. When the tablets comprise such other pharmaceutical excipients, the polyelectrolyte complex of the present invention represents in embodiments, more than about 50, 60, 70, 80, or 90% (w/w) of the total amount of excipients in the tablet.

Non-toxic excipients include but are not limited to:

Antiadherents

Antiadherents are used to reduce the adhesion between the powder (granules) and the punch faces and thus prevent sticking to tablet punches. They are also used to help protect tablets from sticking. Most commonly used is magnesium stearate.

Binders

Binders hold the ingredients in a tablet together. Binders ensure that tablets and granules can be formed with required mechanical strength, and give volume to active dose tablets. Binders include saccharides and their derivatives: disaccharides: sucrose, lactose; polysaccharides and their derivatives: starches, cellulose or modified starch or cellulose such as microcrystalline cellulose and cellulose ethers such as hydroxypropyl cellulose (HPC); sugar alcohols such as xylitol, sorbitol or maltitol. Binders also include protein: gelatin; synthetic polymers: polyvinylpyrrolidone (PVP), polyethylene glycol (PEG).

Binders are classified according to their application:

Solution binders are dissolved in a solvent (for example water or alcohol can be used in wet granulation processes). Examples include gelatin, cellulose, cellulose derivatives, polyvinylpyrrolidone, starch, sucrose and polyethylene glycol. Dry binders are added to the powder blend, either after a wet granulation step, or as part of a direct powder compression (DC) formula. Examples include cellulose, methyl cellulose, polyvinylpyrrolidone and polyethylene glycol.

Coatings

The tablets (dragées, particles) can be coated. The coating of solid oral dosage forms is well-known to the skilled person. Types of pharmaceutical coatings include film-coating, sugar-coating and enteric-coating as well as other types of coatings. The coating will be chosen depending on the nature of the particular active ingredient in the dosage form and the desired release profile/characteristics.

Tablet coatings protect tablet ingredients from deterioration by moisture in the air and make large or unpleasant-tasting tablets easier to swallow. For most coated tablets, a cellulose ether hydroxypropyl methylcellulose (HPMC) film coating is used which is free of sugar and potential allergens. Occasionally, other coating materials are used, for example synthetic polymers, shellac, maize protein zein or other polysaccharides. Capsules are coated with gelatin. Enteric coatings control the rate of drug release and determine where the drug will be released in the digestive tract.

Disintegrants

Disintegrants expand and dissolve when wet causing the tablet to break apart in the digestive tract, releasing the active ingredients for absorption. They ensure that when the tablet is in contact with water, it rapidly breaks down into smaller fragments, facilitating dissolution. Examples of disintegrants include without limitations: crosslinked polymers: crosslinked polyvinylpyrrolidone (crospovidone), crosslinked sodium carboxymethyl cellulose (croscarmellose sodium). The modified starch sodium such as starch glycolate.

Fillers and Diluents

Fillers fill out the size of a tablet or capsule, making it practical to produce and convenient for the consumer to use. By increasing the bulk volume, the fillers make it possible for the final product to have the proper volume for patient handling. A good filler must be inert, compatible with the other components of the formulation, non-hygroscopic, relatively cheap, compactible, and preferably tasteless or pleasant tasting. Plant cellulose (pure plant filler) is a popular filler in tablets or hard gelatin capsules. Dibasic calcium phosphate is another popular tablet filler. A range of vegetable fats and oils can be used in soft gelatin capsules. Other examples of fillers include: lactose, sucrose, glucose, mannitol, sorbitol, calcium carbonate, and magnesium stearate.

Flavours

Flavours can be used to mask unpleasant tasting active ingredients and improve the acceptance that the patient will complete a course of medication. Flavourings may be natural (e.g. fruit extract) or artificial. For example, to improve: a bitter product—mint, cherry or anise may be used; a salty product—peach, apricot or liquorice may be used; a sour product—raspberry or liquorice may be used; an excessively sweet product—vanilla may be used.

Colours

Colours are added to improve the appearance of a formulation. Colour consistency is important as it allows easy identification of a medication.

Lubricants

Lubricants prevent ingredients from clumping together and from sticking to the tablet punches or capsule filling machine. Lubricants also ensure that tablet formation and ejection can occur with low friction between the solid and die wall. Common minerals like talc or silica, and fats, e.g. vegetable stearin, magnesium stearate or stearic acid are the most frequently used lubricants in tablets or hard gelatin capsules. Lubricants are agents added in small quantities to tablet and capsule formulations to improve certain processing characteristics.

There are three roles identified with lubricants: 1) True Lubricant Role, to decrease friction at the interface between a tablet's surface and the die wall during ejection and reduce wear on punches & dies; 2) Anti-adherent Role to prevent sticking to punch faces or in the case of encapsulation, lubricants; Prevent sticking to machine dosators, tamping pins, etc. 3. Glidant Role, to enhance product flow by reducing interparticulate friction.

The major types of lubricants are 1) Hydrophilic, which are generally poor lubricants, no glidant or anti-adherent properties and 2) Hydrophobic, which are most widely used lubricants in use today. Hydrophobic lubricants are generally good lubricants and are usually effective at relatively low concentrations. Many also have both anti-adherent and glidant properties. For these reasons, hydrophobic lubricants are used much more frequently than hydrophilic compounds. Examples include magnesium stearate.

Glidants

Glidants are used to promote powder flow by reducing interparticle friction and cohesion. These are used in combination with lubricants as they have no ability to reduce die wall friction. Examples include fumed silica, talc, and magnesium carbonate.

Preservatives

Some typical preservatives used in pharmaceutical formulations are antioxidants like vitamin A, vitamin E, vitamin C, retinyl palmitate, and selenium; the amino acids cysteine and methionine; citric acid and sodium citrate; synthetic preservatives like the parabens: methyl paraben and propyl paraben.

Sorbents

Sorbents are used for tablet/capsule moisture-proofing by limited fluid sorbing (taking up of a liquid or a gas either by adsorption or by absorption) in a dry state.

Sweeteners

Sweeteners are added to make the ingredients more palatable, especially in chewable tablets such as antacid or liquids like cough syrup. Sugar can be used to mask unpleasant tastes or smells.

The above dosage forms can be produced by methods well known to those of skill in the art. For example two basic techniques are used to granulate powders for compression into a tablet: wet granulation and dry granulation. Powders that can be mixed well do not require granulation and can be compressed into tablets through direct compression.

Wet Granulation

Wet granulation is a process of using a liquid binder to lightly agglomerate the powder mixture. The amount of liquid has to be properly controlled, as over-wetting will cause the granules to be too hard and under-wetting will cause them to be too soft and friable. Aqueous solutions have the advantage of being safer to deal with than solvent-based systems but may not be suitable for drugs which are degraded by hydrolysis. In wet granulation, the active ingredient and excipients are weighed and mixed. The wet granulate is prepared by adding the liquid binder-adhesive to the powder blend and mixing thoroughly. Examples of binders/adhesives include without limitations aqueous preparations of cornstarch, natural gums such as acacia, cellulose derivatives such as methyl cellulose, gelatin, and povidone. The damp mass is then screened through a mesh to form pellets or granules, and the granulation is dryed, for example in a conventional tray-dryer or fluid-bed dryer, which are most commonly used for this purpose. After the granules are dried, they are passed through a screen of smaller size than the one used for the wet mass to create granules of uniform size. Low shear wet granulation processes use very simple mixing equipment, and can take a considerable time to achieve a uniformly mixed state. High shear wet granulation processes use equipment that mixes the powder and liquid at a very fast rate, and thus speeds up the manufacturing process. Fluid bed granulation is a multiple-step wet granulation process performed in the same vessel to pre-heat, granulate, and dry the powders, and allows close control of the granulation process.

Dry Granulation

Dry granulation processes create granules by light compaction of the powder blend under low pressures. The compacts so-formed are broken up gently to produce granules (agglomerates). This process is often used when the product to be granulated is sensitive to moisture and heat. Dry granulation can be conducted on a tablet press using slugging tooling or on a roll press called a roller compactor. Dry granulation equipment offers a wide range of pressures to attain proper densification and granule formation. Dry granulation is simpler than wet granulation, therefore the cost is reduced. However, dry granulation often produces a higher percentage of fine granules, which can compromise the quality or create yield problems for the tablet. Dry granulation requires drugs or excipients with cohesive properties, and a ‘dry binder’ may need to be added to the formulation to facilitate the formation of granules.

Granule Lubrication

After granulation, a final lubrication step is used to ensure that the tableting blend does not stick to the equipment during the tableting process. This usually involves low shear blending of the granules with a powdered lubricant, such as magnesium stearate or stearic acid.

Manufacture of the Tablets

Whatever process is used to make the tableting blend, the process of making a tablet by powder compaction is very similar. First, the powder is filled into the die from above. The mass of powder is determined by the position of the lower punch in the die, the cross-sectional area of the die, and the powder density. At this stage, adjustments to the tablet weight are normally made by repositioning the lower punch. After die filling, the upper punch is lowered into the die and the powder is uniaxially compressed to a porosity of between 5 and 20%. The compression can take place in one or two steps (main compression, and, sometimes, pre-compression or tamping) and for commercial production occurs very fast (500-50 msec per tablet). Finally, the upper punch is pulled up and out of the die (decompression), and the tablet is ejected from the die by lifting the lower punch until its upper surface is flush with the top face of the die. This process is simply repeated many times to manufacture multiple tablets.

Common problems encountered during tablet manufacturing operations include poor (low) weight uniformity, usually caused by uneven powder flow into the die; poor (low) content uniformity, caused by uneven distribution of the API in the tableting blend; sticking of the powder blend to the tablet tooling, due to inadequate lubrication, worn or dirty tooling, and sub-optimal material properties; capping, lamination or chipping. Such mechanical failure is due to improper formulation design or faulty equipment operation; capping also occurs due to high moisture content.

Numerous types of active ingredients may be utilized in the context of the dosage form of the present invention, including pharmaceutical drugs (e.g., small-molecule therapeutic or prophylactic agents), a diagnostic agent or reagent, a neutraceutical, biologics (e.g., polypeptides), a peptide, etc. Non-limiting examples of pharmaceutical drug include:

(i) gastrointestinal and liver drugs,

(ii) hematological drugs,

(iii) cardiovascular drugs,

(iv) respiratory drugs,

(v) sympathomimetic drugs,

(vi) cholinomimetic drugs,

(vii) adrenergic, and adrenergic neuron blocking drugs,

(viii) antimuscarinic and antispasmodic drugs,

(ix) skeletal muscle relaxants,

(x) diuretic drugs,

(xi) uterine and antimigraine drugs,

(xii) hormones and hormone antagonists,

(xiii) general anesthetics,

(xiv) sedative and hypnotic drugs,

(xv) antiepileptic drugs,

(xvi) psychopharmacologic agents,

(xvii) analgesic, antipyretic, and anti-inflammatory drugs,

(xviii) histamine and antihistaminic drugs,

(xix) central nervous system stimulants,

(xx) antineoplastic and immunoactive drugs,

(xxi) anti-infectives,

(xxii) parasiticides, and

(xxiii) immunizing agents and allergenic extracts.

The present invention also relates to a method of manufacturing a polyelectrolyte complex of carboxymethyl starch and chitosan, the method comprising coagulating together carboxymethyl starch and chitosan in a solvent. Herein, coagulating means mixing together a solution of carboxymethyl starch and a solution of chitosan under conditions resulting in the coagulation of the desired polyelectrolyte complex. The coagulation is observed because, while both chitosan and carboxymethyl starch are soluble in the solvent, the polyelectrolyte complex is not. Coagulation is herein defined as the process by which molecules of the polyelectrolyte complex aggregate and appear as particles in the solvent. These particles may eventually settle or otherwise be isolated from the solvent.

In embodiments, the coagulating step is carried out in an aqueous medium. In such cases, the coagulating step is carried out by mixing an aqueous solution of carboxymethyl starch and an aqueous solution of chitosan. This can be carried out, for example, at room temperature.

In embodiments, the coagulating step is carried out at a (—NH₃⁺:—COO⁻) ratio ranging from about 1:0.5 to about 0.5:1. In more specific embodiments, it is carried out at about a 1:1 (—NH₃⁺:—COO⁻) ratio.

The method may further comprise isolating the polyelectrolyte complex. As the polyelectrolyte complex is coagulated, it can easily be isolated by methods well known to the skilled person, such as filtration and decantation. Finally, the method may further comprise washing and drying the polyelectrolyte complex.

As used herein, “about” in reference to a numerical value encompasses the typical variation in measuring the value, in an embodiment plus or minus 10% of the numerical value.

Herein, “pharmaceutically acceptable”, such as in “pharmaceutically acceptable carrier”, means physiologically compatible and substantially non-toxic to the subject or biological system to which the particular compound is administered.

### DESCRIPTION OF ILLUSTRATIVE EMBODIMENTS

The present invention is illustrated in further details by the following non-limiting examples.

### Example 1

Preparation of CMS-Chitosan Polyexlectrolyte Complex

### 1. Objectives

The objectives are
(xxiv) to prepare CMS-chitosan polyelectrolyte complex (PEC) and to investigate its performance in drug delivery;
(xxv) to evaluate the influence of chitosan molecular weight on drug release rate;
(xxvi) to compare the drug dissolution from tablets based on anionic water-soluble excipient (CMS) alone, on cationic water-insoluble excipient (chitosan) alone, on physical mixture powder of these two excipients, or on PEC; and
(xxvii) to compare the dissolution profiles of drugs with different charges and solubilities.

As will be seen below, a novel polyelectrolyte complex (PEC) of carboxymethyl starch (CMS) and chitosan was prepared, characterized and tested in vitro as a carrier for oral drug delivery. This PEC, containing 14% (w/w) of chitosan, showed a polymorphism with a lower order degree than those of CMS and of chitosan. Under conditions simulating the gastrointestinal transit, NMR imaging analysis showed slower fluid diffusion inside PEC monolithic tablets than inside CMS tablets. The PEC as appears to be a more suitable drug carrier for colon targeting than CMS, since it can prolong acetaminophen release time from 8 h to 11 h and aspirin release time from 13 h to 30 h. In contrast, chitosan used as a coexcipient accelerated aspirin release from matrices based on a CMS:chitosan physical mixture (i.e., not in a polyelectrolyte complex).

### 2. Materials and Methods

### 2.1. Materials

High amylose corn starch (Nylon VII) is obtained from National Starch (Bridgewater, N.J., USA) and crab shell chitosans are from Marinard Biotech (Rivière-au-Renard, QC, Canada). Acetaminophen is from Sigma-Aldrich (St-Louis, Mo., USA). Metformin (1,1-dimethylbiguanide hydrochloride) is from MP Biomedicals (Solon, Ohio, USA). Aspirin (acetylsalicylic acid) and monochloroacetic acid are from Fisher Scientific (Fair Lawn, N.J., USA). The other chemicals are of reagent grade and used without further purification. Pepsin-free Simulated Gastric Fluid (SGF, pH 1.2) and Pancreatin-free Simulated Intestinal Fluid (SIF, pH 6.8) are prepared following the USP methods (US Pharmacopeia, XXIV, 2000).

### 2.2. Preparation of CMS and Purification of Chitosans

Sodium carboxymethyl starch (CMS) is prepared in aqueous medium from high amylose corn starch as previously described (Mulhbacher, Ispas-Szabo, Lenaerts & Mateescu, 2001; Calinescu et al., 2005), with minor modifications. Briefly, an amount of 70 g of Hylon VII is suspended in 170 mL of distilled water in a Hobart mixer (Vulcan, Canada) at 55° C. Then, 235 mL of 1.5 M NaOH are added for gelatinization under continuous mixing for 30 min. Subsequently, 55 mL of 10 M NaOH and a freshly prepared solution of monochloroacetic acid (45.5 g in 40 mL of distilled water) are added. After 1 h of reaction, a volume of 130 mL of distilled water is added and the slurry is cooled-down to room temperature and neutralized with acetic acid. The CMS is then precipitated from the slurry by gradually adding 600 mL of acetone. After that, the CMS is washed by repeated dispersion in volumes of 1 L of 70% acetone and filtrations until a final conductivity of filtrate decreased at about 50 μS/cm. The CMS mass is again washed three times with acetone, and then dried at 40° C. for 24 h. The obtained powder of sodium form CMS is sieved with a 300 μm screen and stored at room temperature.

Two chitosans of different molecular weights are each purified by solubilization in acetic acid and by filtration as follows: an amount of 20 g of chitosan is solubilized in 350 mL of 0.35 M acetic acid and the volume is adjusted to 2 L with distilled water. The acidic solution is filtered under vacuum through Whatman filter papers (medium 40). Subsequently, the chitosan is precipitated with 0.1 M NaOH under continuous stirring. The mass is washed with distilled water, then with nanopure water (volumes of 2 L) until conductivity of about 200 μS/cm and finally with acetone. The chitosan is dried at 40° C. for 24 h, ground and sieved on a 300 μm screen.

### 2.3. Preparation of CMS-Chitosan PEC

A CMS-chitosan polyelectrolyte complex (PEC) is prepared by coagulation of CMS and chitosan-700 in aqueous medium at room temperature. Essentially, 1 g of chitosan-700 is solubilized in 44 mL of 0.1 M HCl, and the volume is adjusted to 150 mL with distilled water. A 1% solution of CMS is prepared by solubilizing 6 g of CMS in 600 mL of distilled water. The precipitation occurred under vigorous mixing by adding the solution of polycation (chitosan-700) to that of polyanion (CMS) at 1:1 ratio (—NH₃⁺:—COO⁻), with a final pH about 5. The PEC, containing 14% (w/w) of chitosan-700, is washed and dried with acetone by the same procedure as for CMS.

### 2.4. Physical and Chemical Characterizations of Excipients

The degree of substitution (DS) of CMS is determined by back-titration as previously described (Assaad & Mateescu, 2010). Briefly, 300 mg of protonated CMS (n=3) are solubilized in 20 mL of 0.05 M NaOH and then the excess of NaOH is titrated with 0.05 M HCl using phenolphthalein as indicator. The blank (20 mL of NaOH) is also titrated by the same method. The amount of —COOH groups and the DS are calculated by using the following equations (Stojanovic, Jeremic, Jovanovic & Lechner, 2005):

$$\begin{matrix}{n}_{COOH} = ( {V}_{b} - V ) \times {C}_{HCl} & ( 1 ) \\ DS = \frac{( 162 \times {n}_{COOH} )}{m - 58 \times {n}_{COOH}} & ( 2 )\end{matrix}$$

where V_{b} (mL) is the volume of HCl used for the titration of the blank; V (mL) is the volume of HCl used for the titration of the sample; C_{HCl} (mol/L) is the concentration of HCl; 162 (g/mol) is the molar mass of glucose unit; 58 (g/mol) is the increase in the mass of glucose unit by substitution with one carboxymethyl group, and m (g) is the mass of dry sample.

The degree of deacetylation (DDA) of each chitosan is determined by acid-base titration. An amount of 150 mg of chitosan is solubilized in 20 mL of 0.1 M HCl and the volume is completed to 200 mL with distilled water. A titration is done with 0.1 M NaOH and the pH and the conductivity are recorded. The DDA is calculated following the method and the equation given by Broussignac (1968) and Muzzarelli (1977):

$$DDA ( \% ) = \frac{203 \times ( {v}_{2} - {v}_{1} ) \times M \times 100}{m + 42 \times ( {v}_{2} - {v}_{1} ) \times M} \qquad ( 3 )$$

where V₁ and V₂ are the volumes of NaOH solutions corresponding to the two inflexion points of the curve obtained by titration; M is the concentration of NaOH (mol/L); m is the weight of chitosan (g); 203 (g/mol) is the molar mass of acetylated unit, and 42 (g/mol) is the difference between molar mass of acetylated unit and that of deacetylated unit.

The molecular weights of chitosans are determined by viscometric method, using experimental reported viscometric constants data (Knaul, Kasaai, Bui & Creber, 1998; Kasaai, 2007). Samples are dissolved in a solution containing 0.1 M acetic acid and 0.2 M sodium chloride for chitosan-400 and in a solution containing 0.2 M acetic acid and 0.1 M sodium acetate for chitosan-700. The viscosities of chitosan solutions with different concentrations (0.07-0.7%) are measured by using an electronic viscometer (Viscosity Monitoring and Control Electronics, Medford, Mass., USA). The temperature is adjusted at 25° C. for chitosan-400 and at 30° C. for chitosan-700.

The data on viscosities and concentrations are used to calculate the reduced viscosities. Plotting reduced viscosities against chitosan concentrations gives the intrinsic viscosity ([η]) by extrapolation of the straight line obtained by linear regression to zero concentration. The average molecular weight (M) of chitosan is calculated from the intrinsic viscosity by Mark-Houwink-Sakurada's empirical equation:

[η]=kM^{α} (4)

where k (dL/g) and a (dimensionless) are constants that depend on the solvent-polymer system.

The Fourier Transform Infrared spectra (FTIR) of samples are recorded from 4000 to 400 cm⁻¹, at 2 cm⁻¹, resolution with a total of 32 scans by using a Nicolet 4700 spectroscopy (Madison, Wis., USA). To prepare the pellets, homogenous mixtures of dried KBr (67 mg) and of polymer powders (3 mg) are compressed at 3 tonnes (Carver, Wabash, Ind., USA) in flat-faced punches with 12 mm diameter.

The polymorphism of samples is evaluated by X-ray diffractometer (XRD, Siemens D5000, Munich, Germany) at 1.789 Å wavelength. The original XRD spectra, recorded between 5 and 50 degrees (2-theta), are treated using Excel software (regression type: moving average, period 10).

The thermogravimetric analyses are carried out in platinum crucible at a heating rate of 10° C./min between 25 and 900° C. under nitrogen atmosphere (flow rate 100 ml/min). A Seiko TG/DTA 6200 (Japan) instrument is used and the alumina is taken as reference material.

The morphology of the sample particles is examined by a Hitachi (S-4300SE/N) scanning electron microscopy with variable pressure (Hitachi High Technologies America, Pleasanton, Calif., USA) at voltage of 15 kV and magnification of 100× and 500×. Samples are mounted on metal stubs and sputter-coated with gold.

The density of the polymer powders is determined according to the (616) USP method, using a Vankel tapped density tester (Varian, N.C., USA).

### 2.5. Preparation of Tablets

Monolithic tablets (200 mg, 20% w/w loading) are obtained by direct compression (2.5 tonnes) of a homogenous mixture of excipient and drug (acetaminophen, metformin or aspirin) powders. The unloaded (drug-free) tablets of 200 mg are prepared with excipient only. Flat-faced punches with 9.6 mm diameter and a Carver hydraulic press are used.

Dry-coated (DC) tablets (200 mg, 20% w/w loading) are prepared with a core consisting in a homogenous mixture of drug (40 mg) and excipient (40 mg) and compressed in a 7 mm cylinder outfit. This core is then dry coated with 120 mg of excipient, giving tablet of about 9.6 mm diameter and 2.1 mm thickness after compression.

### 2.6. Nuclear Magnetic Resonance (NMR) Imaging Tests

NMR imaging analyses are carried out at 37° C. with a Bruker Avance-400 NMR spectrometry (Germany) as previously reported (Wang, Ravenelle & Zhu, 2010). A standard spin-echo pulse sequence (90-τ-180-τ-Acquisition) is used to obtain spin density images of the unloaded tablets (n=3) in a NMR tube (20 mm diameter) containing 20 mL of dissolution media (SGF or SIF). A slice of 0.5 mm in thickness is selected either perpendicular or parallel to the main magnetic field (axial axis). Eight scans are accumulated with a field of view of 2 cm and an in-plane resolution of 156 μm. An echo time of 3 ms and a repetition time of 1 s are fixed, leading to an acquisition time of about 17 min for each image. Each tablet is first incubated for 2 h in SGF and then in SIF until the end of the test. The percentage of axial and radial swelling is calculated by comparison to the initial dimension of tablet.

### 2.7. In Vitro Dissolution Tests

The in vitro dissolution tests are carried out at 100 rpm and 37° C. in an USP dissolution apparatus II (Distek 5100, North Brunswick, N.J., USA) coupled with an UV spectrophotometer (Hewlett Packard 8452A, USA). The tablets (n=3) are incubated in SGF (1 L) for 2 h and then in SIF (1 L) up to complete release. The drug release from tablets is evaluated by measuring the absorbance at the appropriate wavelength (acetaminophen at 244 nm, metformin at 218 nm, and aspirin at 246 nm).

### 3. Results and Discussion

### 3.1. Characterization of the Excipients

The degree of substitution of carboxymethyl starch (CMS) determined by the back-titration method is about 0.14, representing the average number of carboxymethyl groups per glucose unit. The degree of deacetylation of chitosans determined by acid-base titration are about 80% and the approximate molecular weights determined by Mark-Houwink-Sakurada method are about 400 kDa for chitosan-400 and 700 kDa for chitosan-700.

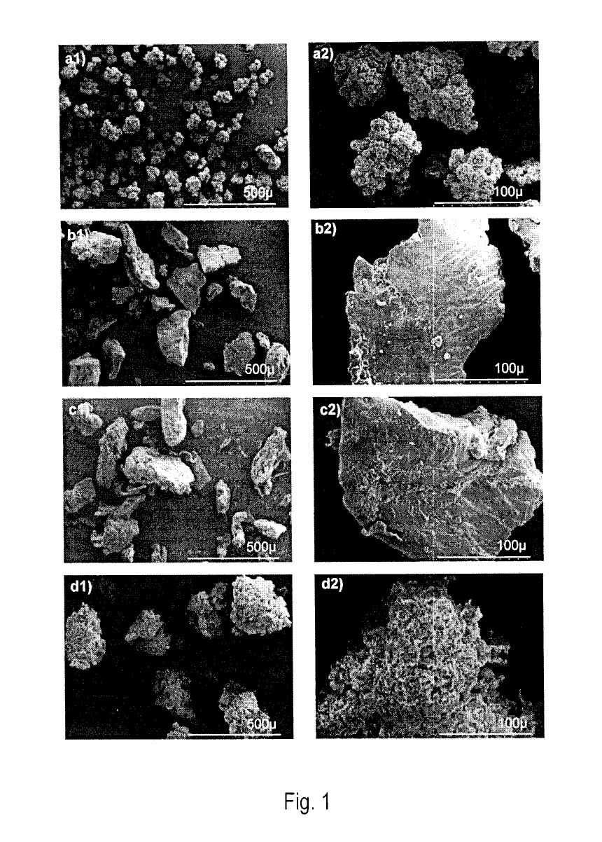

Scanning electron microscopy micrographs showed that chitosan particles are compact, whereas those of CMS and PEC are porous (FIG. 1). The morphology of the polyelectrolyte complex (FIGS. 1d1 and 1d2) appeared homogenous, indicating a uniform distribution and a good compatibility between CMS and chitosan.

Chitosan-400 and chitosan-700 showed the highest tapped densities (0.61 and 0.64 g/mL, respectively) due to their compact morphology, whereas PEC showed the lowest density (0.20 mg/mL) due to its higher granulometry and porosity (FIG. 1). Intermediate density (0.36 mg/L) is found for CMS.

### 3.2. CMS-Chitosan Interactions and Preparation of PEC

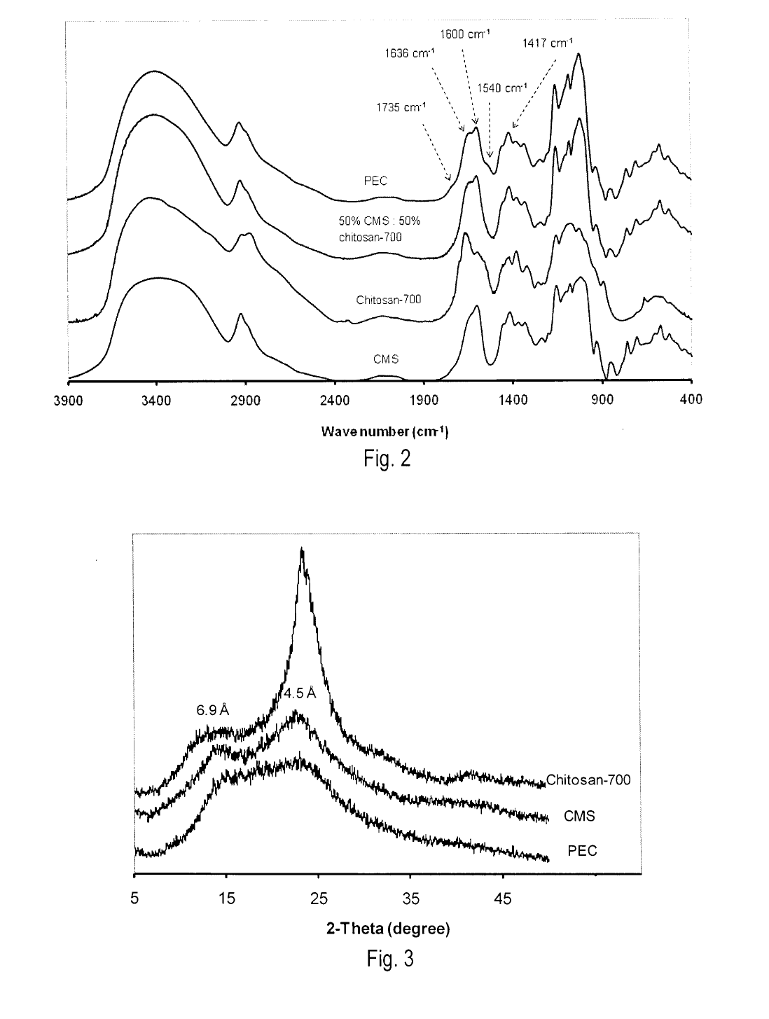

When the chitosan-700 solution is added to the CMS solution, immediate coagulation and precipitation occurred. This suggests effective interactions between functional groups of CMS and of chitosan-700 with possible partial charges neutralization, leading to the formation of a polyelectrolyte complex. To verify this hypothesis, the products are characterized by FTIR spectroscopy, by X-ray diffractometry (XRD) and by thermogravimetry (TGA) (FIGS. 2, 3 and 4).

The FTIR spectrum of CMS (FIG. 2) presents two characteristic bands at 1603 and 1417 cm⁻¹. They are attributed respectively to asymmetrical and symmetrical stretching vibration of —COO⁻ groups. The bands at 2930 and 1643 cm⁻¹ are assigned respectively to C—H stretching and to O—H groups.

The spectrum of chitosan-700 shows characteristic absorption bands of chitosan at 1653 and 1597 cm⁻¹ ascribed to —CONH₂ stretching vibrations, and two bands at 2922 and 2876 cm⁻¹ due to C—H stretching. The bands at 1417 and 1376 cm⁻¹ are assigned to the C—H symmetrical deformation mode.

The polyelectrolyte complex (PEC) shows a spectrum similar to that of 50% CMS:50% chitosan-700, with bands at about 2923-2880, 1636, 1600, 1417 and 1376 cm⁻¹. This indicates the presence of both CMS and chitosan in the PEC. The weak shoulders at around 1735 and 1540 cm⁻¹ for PEC obtained at pH 5 could be assigned respectively to —COOH and —NH₃⁺ groups. These shoulders suggest that interactions between CMS and chitosan in the PEC may occur via hydrogen bonds (—OH, —COOH) or ionic interactions (—COO⁻, NH₃⁺).

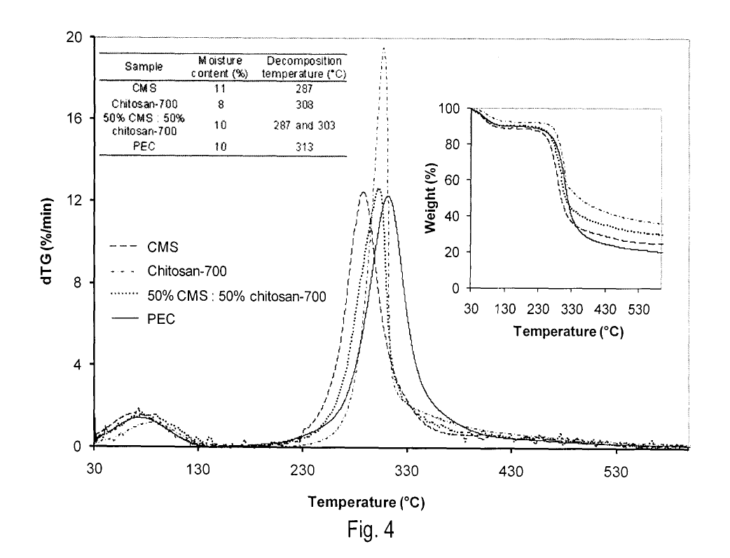

The XRD pattern (FIG. 3) of CMS shows the two characteristic peaks at 6.9 and 4.5 Å, indicating a V-type single helix structure. The pattern of chitosan-700 shows characteristic crystalline peaks at around 6.9 and 4.4 Å (major one), fitting well with the typical XRD pattern of chitosan.

The order degree of the PEC is definitely lower to those of CMS and chitosan-700. The suppression of crystalline peak of chitosan-700 at 4.4 Å and the broad amorphous pattern of the PEC indicate a good compatibility and strong interactions between CMS and chitosan with a complete dispersion of chitosan chains. These intermolecular interactions could prevent macromolecules to crystallize individually as reported for some interpolymer complexes.

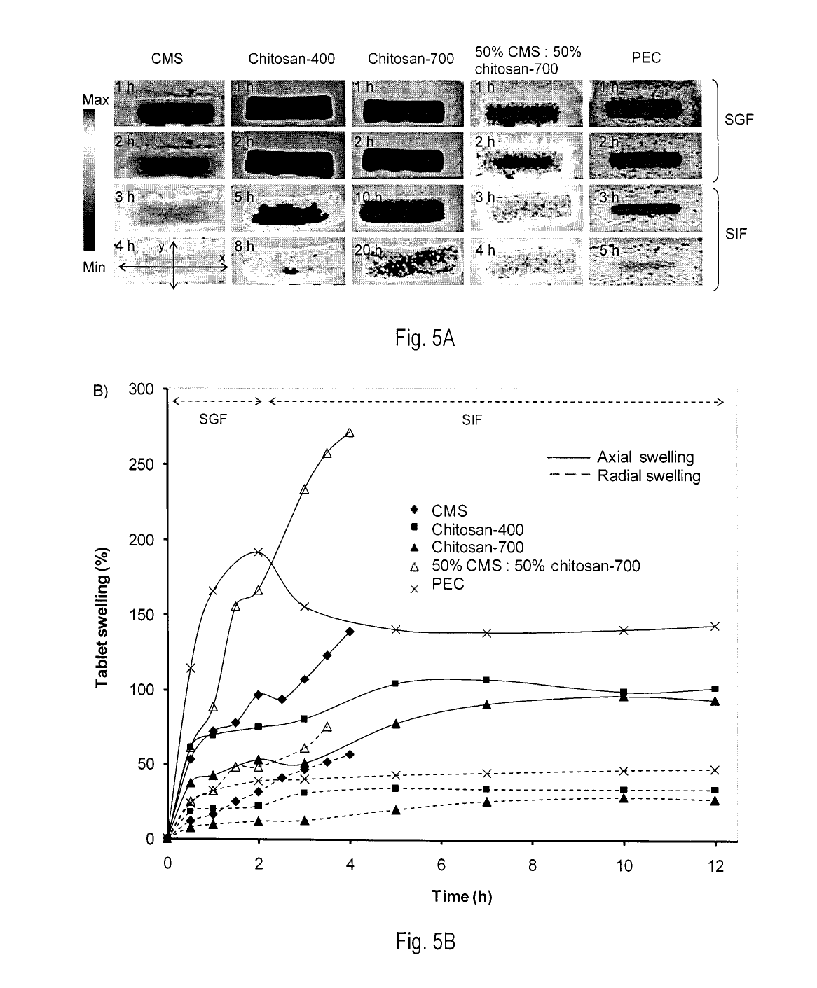

The TGA results (FIG. 4) show relatively lower moisture content for chitosan-700 than for CMS and PEC, maybe due to higher hydrogen association of chitosan chains. The 50% CMS:50% chitosan-700 shows a nonsymmetrical dTG peak with a weak shoulder at around 287° C. and a maximum at 303° C., indicating the presence of two components. The difference of decomposition temperatures between CMS (287° C.) and chitosan-700 (308° C.) seems not enough to identify two separate peaks for the dry powder mixture of these two polymers. Differing from 50% CMS:50% chitosan-700, the PEC presented a symmetrical dTG peak and the highest decomposition temperature (313° C.).

Overall, these results suggest a good compatibility between CMS and chitosan, a strong interaction between the chains of these two polymers, and the formation of a homogenous polyelectrolyte complex.

### 3.3. Examination of Tablets Hydration and Swelling by NMR

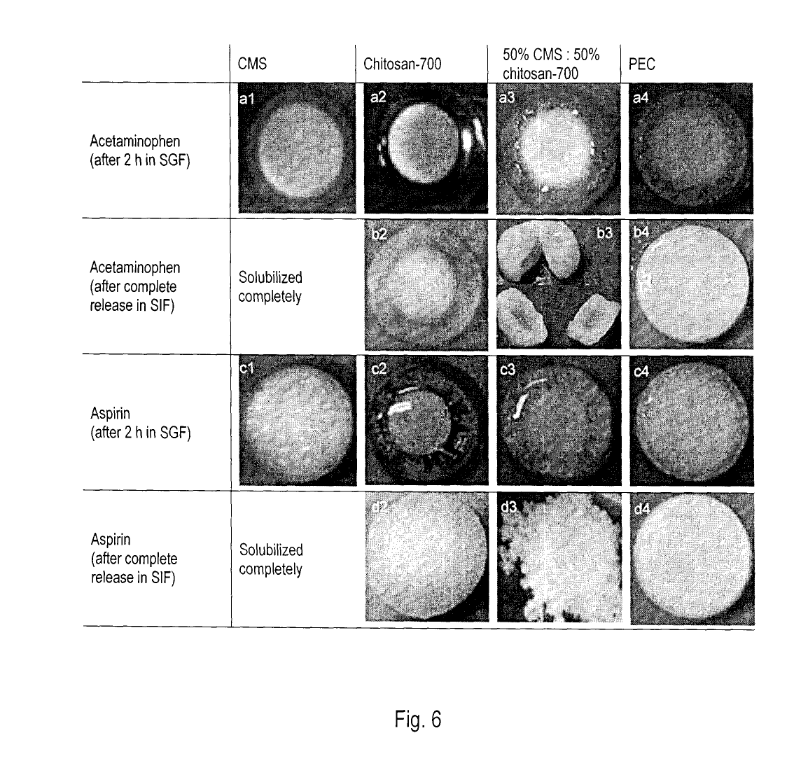

Water penetration into unloaded tablets (FIG. 5A) and the axial and radial swelling (FIG. 5B) are followed by NMR imaging in SGF for 2 h and then in SIF to simulate the gastrointestinal transit. The location where the water concentration matches ⅙ of the maximal concentration corresponding to free fluid (SGF or SIF) is considered as the front of fluid diffusion inside the tablets.

For all tablets, the axial swelling is higher than radial swelling (FIG. 5B). This may be explained by the formation of flat oriented particles in tablet after axial compression of polymer powders. Upon tablet hydration, the stress resulting from compression is released, leading to a higher swelling in the direction where compression force is applied.

After 2 h in SGF, the CMS tablet still showed a dry core (dark gray) with a partial penetration of SGF and formation of a gel network in the outer layer (white-pale gray) (FIG. 5A). In acidic medium (SGF, pH 1.2), the outer layer carboxylate groups (—COONa) are converted to carboxyl groups (—COOH), thus reducing the solubility of the CMS excipient and limiting the gastric fluid penetration into the tablets. When tablets are transferred to SIF (pH 6.8), the fluid advanced rapidly to the core which became hydrated within 2 h in this neutral medium. The protonation acquired in SGF is lost and the —COOH groups turn into their salt form (—COOK), increasing thus the solubility of the excipient and accelerating intestinal fluid advancement to the core of the tablet. The axial and radial swelling of CMS tablet increase relatively fast, reaching 150% and 60%, respectively, after 4 h of incubation (FIG. 5B).

The diffusion of fluid (SGF or SIF) into the chitosan (chitosan-400 and chitosan-700) tablets is slower than into the CMS tablets (FIG. 5A). A gel network is developed by chitosan in SGF due to the protonation of amino groups exposed to the acid medium. In SIF, the tablet size is stabilized (FIG. 5B) due to chitosan insolubility in neutral medium while an anisotropic fluid diffusion is observed (FIG. 5A). For chitosan-400 the core is almost completely hydrated after 8 h of incubation, whereas for chitosan-700 the core still showed dry regions even after 20 h. Thus, chitosan-700 with a higher molecular weight seems to provide a thicker (more substantial) outer layer gel than chitosan-400.

The tablets of 50% CMS:50% chitosan-700 mixture showed the fastest fluid diffusion (FIG. 5A) and the highest swelling (FIG. 5B). The gel network formed in SGF is less substantial than that formed with chitosan tablets due to close neighboring of CMS in the mixture. In SIF, the chitosan would be deprotonated, whereas the CMS would be converted to the salt form, triggering a higher in situ hydration of tablet previously swollen in SGF.

The tablets of PEC presented slower fluid diffusion than CMS and 50% CMS:50% chitosan-700 tablets, particularly in SIF (FIG. 5A). Without being bound to a particular theory, this suggests that association of CMS and chitosan at molecular level as PEC favors more interactions between these two compounds than in physical mixture of powders. A more extensive swelling occurred in the first two hours of incubation in SGF due to the protonation and the hydration of chitosan within the PEC. When SGF is changed to SIF, the size of PEC tablets is reduced due to the deprotonation and dehydration of chitosan chains in neutral medium, indicating a higher interaction between chitosan and CMS than that between CMS and water. The shape of tablets is after that as stable as those of the chitosan, despite the low ratio (14%) of chitosan in PEC. This is an important aspect and can be related to the insolubility of chitosan in neutral medium and to a lower tendency of CMS to swell when intimately complexed with chitosan.

### 3.4. In Vitro Dissolution Tests

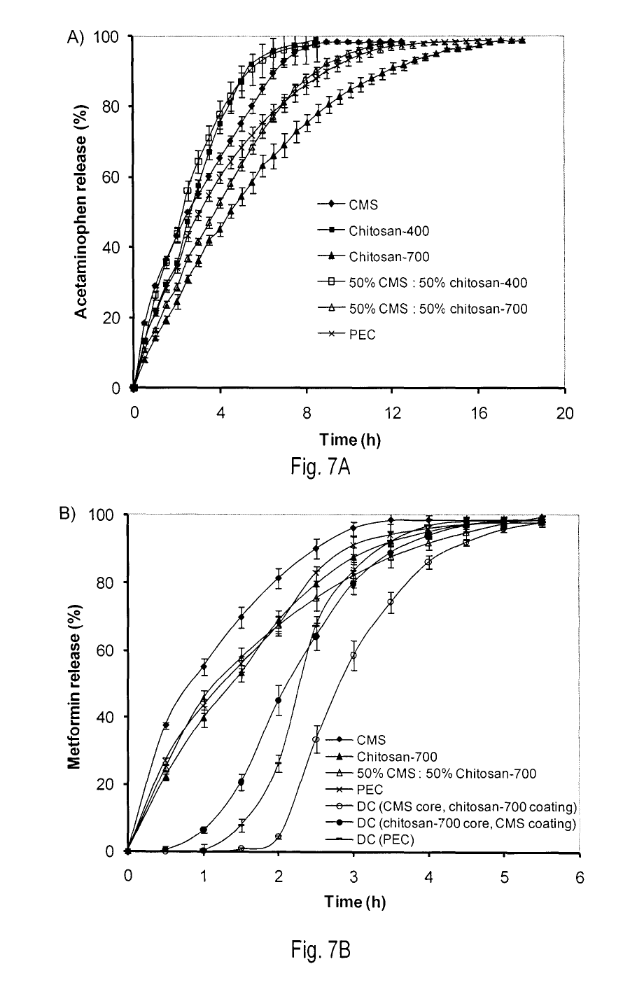

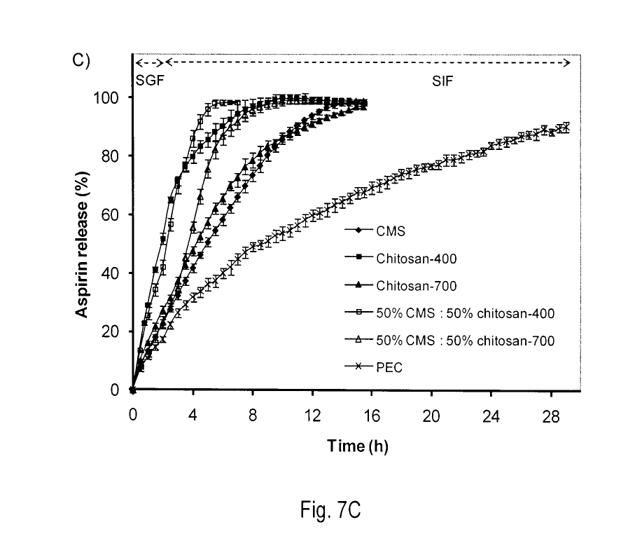

All dissolution tests, except for metformin formulated in monolithic tablets, are followed first in SGF for 2 h and then in SIF until complete drug release, simulating thus the gastrointestinal transit. The shape of tablets and the dissolution profiles of acetaminophen, metformin and aspirin are presented in FIGS. 6 and 7. Unless otherwise specified, the tablets (200 mg) used for dissolution are monolithic.

The release rates of acetaminophen from chitosan-400 and 50% CMS:50% chitosan-400 matrices are higher than from CMS matrix, whereas the release rates from chitosan-700 and 50% CMS:50% chitosan-700 matrices are lower than from CMS matrix (FIG. 7 A). That is why the chitosan-700 is chosen to prepare the PEC. In addition, the 75% CMS:25% chitosan-700 matrix showed almost the same release rate as CMS. It seems that a molecular weight of 700 kDa rather than 400 kDa and an adequate ratio in dry blends favor a longer drug release time through chitosan action.

Although the chitosan-700 matrix showed the lowest release rate, chitosan alone does not seem suitable for controlled drug release, because the transformation of the gel developed in SGF (FIG. 6 a2) to a semi-solid form (FIG. 6 b2) that limits the diffusion of SIF into the tablet makes the release slow (FIG. 7 A). It is worthwhile to note that the solid core of tablet is still compact and insoluble even after the complete acetaminophen release (FIG. 6 b2).

The faster release from tablets based on CMS:chitosan-700 powder mixture compared to that from those with chitosan-700 as only excipient, indicates that the CMS favors the tablet hydration and accelerates the diffusion of SIF into the tablets. These results are in agreement with those obtained by NMR imaging (FIG. 5A). At the end of the dissolution tests, the tablets based on a mixture of CMS and chitosan powders appeared as a water-insoluble empty shell (FIG. 6 b3) with a crust still containing a mixture of these two polymers as confirmed by FTIR analysis (not shown).

The release rate of acetaminophen from PEC matrix is comparable to that from 50% CMS:50% chitosan-700 matrix (FIG. 7 A). This is an interesting advantage for PEC which contains only 14% (w/w) of chitosan-700, considering the higher cost of chitosan compared to that of CMS.

Metformin is a freely soluble drug (US Pharmacopeia, 2000) and its release from hydrophilic excipients is usually fast. Neither CMS nor chitosans, separately or in association, are able to control the release of metformin in monolithic dosage form (FIG. 7 B). Dry coated (DC) tablets can delay this release in SGF, especially when the outer part of tablet is based on chitosan-700. However, when the fluid reached the inner core of the tablets the release is accelerated. The dissolution profiles of metformin from tablets based on chitosan-400 are similar to those based on chitosan-700, but with higher release rate. The dry coating formulation can be of interest, considering the very high hydrosolubility of metformin and the fact that the release is undesired in stomach.

For aspirin, which is slightly soluble in water (US Pharmacopeia, 2000), the higher the molecular weight of chitosan, the lower is the release rate (FIG. 7 C). CMS and chitosan-700 provided a low release of aspirin in SGF and a long sustained release in SIF. Probably, this is due to the interactions of carboxyl groups of aspirin with carboxyl groups and hydroxyl groups of CMS, and to the formation of consistent outer layer gel network in tablets based on chitosan (FIG. 6 c2). As with acetaminophen, the dissolution of aspirin from chitosan-700 matrix after 80% of release is reduced probably due to the formation of chitosan insoluble outer layer in SIF (FIG. 6 d2). The matrices based on CMS:chitosan mixture showed an accelerated release of aspirin compared to those based on individual excipient (CMS or chitosan). Similar to what is observed with acetaminophen, a water-insoluble excipient residue is still present after complete release of aspirin (FIG. 6 d3), indicating the presence of CMS-chitosan interactions. A sustained release of aspirin, over more than 30 h, is observed with PEC (FIG. 7 C). This time release is markedly longer than (t_{90%}) obtained with 50% CMS:50% chitosan-700 (6.5 h), CMS (11 h) or chitosan-700 (11.5 h).

Taken together, these results showed the advantage of PEC for monolithic formulations. The PEC excipients, containing only 14% of chitosan-700, can afford controlled release of acetaminophen and aspirin. Moreover, the tablets of the PEC are homogenous and less swellable than those of 50% CMS:50% chitosan-700 (FIGS. 5B and 6).

The CMS-chitosan polyelectrolyte complex (PEC) showed a polymorphism with a lower order degree than those of carboxymethyl starch (CMS) and of chitosan-700. The fluid (SGF or SIF) diffusion and the swelling are lower with PEC tablets than with those based on CMS:chitosan-700 powder mixture. The PEC provided a controlled release of acetaminophen and a markedly slower sustained release of aspirin than that provided by CMS or chitosan-700, making this excipient favorable to colon targeting.

Chitosan at relatively low molecular weight (chitosan-400, 400 kDa) is not able to afford a long release time for any of the three tracer drugs (metformin, acetaminophen and aspirin). CMS and chitosan-700 matrices showed a fast release of metformin, a controlled release of acetaminophen and a sustained release of aspirin. This indicates that, when using CMS or chitosan-700 alone, the drug solubility has a major influence on release rate irrespective of the charge of drug and of excipient. The low hydration of chitosan and its insolubility in neutral medium can be a limitation for drug delivery with this excipient alone, but it can be an advantage in the case of PEC. Adding an adequate amount of chitosan with an appropriate molecular weight to the formulations based on CMS can prolong the release time of acetaminophen. Contrarily, the aspirin release from CMS matrix is accelerated when chitosan is added as coexcipient.

While preferred embodiments have been described above and illustrated in the accompanying drawings, it will be evident to those skilled in the art that modifications may be made without departing from this disclosure. Such modifications are considered as possible variants comprised in the scope of the disclosure. In the claims, the word “comprising” is used as an open-ended term, substantially equivalent to the phrase “including, but not limited to”. The singular forms “a”, “an” and “the” include corresponding plural references unless the context clearly dictates otherwise.

1. A carboxymethyl starch and chitosan polyelectrolyte complex, wherein the carboxymethyl starch is a high amylose carboxymethyl starch.

2. The polyelectrolyte complex of claim 1, wherein the carboxymethyl starch is a carboxymethyl starch salt.

3. The polyelectrolyte complex of claim 2, wherein the carboxymethyl starch salt is sodium carboxymethyl starch or potassium carboxymethyl starch.

4. (canceled)

5. The polyelectrolyte complex of claim 2, wherein the carboxymethyl starch salt is partially protonated.

6. The polyelectrolyte complex of claim 1, wherein the degree of substitution of the carboxymethyl starch is between about 0.03 and about 2.

7. The polyelectrolyte complex of claim 6, wherein the degree of substitution of the carboxymethyl starch is about 0.14.

8. The polyelectrolyte complex of claim 1, wherein the degree of deacetylation of the chitosan is about 65% or more.

9. The polyelectrolyte complex of claim 8, wherein the degree of deacetylation of the chitosan is about 80%.

10. The polyelectrolyte complex of claim 1, wherein the molecular weight of the chitosan is about 100 kDa or more.

11. The polyelectrolyte complex of claim 10, wherein the molecular weight of the chitosan is between about 400 and about 700 kDa.

12. The polyelectrolyte complex of claim 11, wherein the molecular weight of the chitosan is about 700 kDa.

13. The polyelectrolyte complex of claim 1, wherein —NH₃⁺ groups of the chitosan and —COO⁻ groups of the carboxymethyl starch are present in a (—NH₃⁺:—COO⁻) ratio ranging from about to about 1:0.5 to about 0.5:1.

14. The polyelectrolyte complex of claim 13, wherein the —NH₃⁺ groups of the chitosan and the —COO⁻ groups of the carboxymethyl starch are present in a 1:1 (—NH₃⁺:—COO⁻) ratio.

15-19. (canceled)

20. An active ingredient carrier comprising the polyelectrolyte complex of claim 1.

21. A solid oral dosage form comprising the polyelectrolyte complex of claim 1 and an active ingredient.

22. The dosage form of claim 21, further comprising an additional pharmaceutically acceptable excipient.

23. A method of manufacturing a carboxymethyl starch and chitosan polyelectrolyte complex, the method comprising coagulating together carboxymethyl starch and chitosan in a solvent, wherein the carboxymethyl starch and the chitosan are as defined in claim 1.

24. (canceled)

25. The method of claim 23, wherein said coagulating is carried out by mixing an aqueous solution of carboxymethyl starch and an aqueous solution of chitosan.

26. (canceled)

27. The method of claim 23, wherein said coagulation is carried out at a (—NH₃⁺:—COO⁻) ratio ranging from about 1:0.5 to about 0.5:1.

28. The method of claim 27, wherein said coagulation is carried out at a 1:1 (—NH₃⁺:—COO⁻) ratio.

29-30. (canceled)
